(19) United States
(12) Patent Application Publication
(10) Pub. No.: US 2023/0102037 A1
(43) Pub. Date: Mar. 30, 2023

(54) KIT FOR DETECTING FOOT-AND-MOUTH DISEASE VIRUS AND DETECTION METHOD THEREOF

(71) Applicant: AFTC of Shanghai Customs, Shanghai (CN)

(72) Inventors: Junxin XUE, Shanghai (CN); Yingzheng LIN, Shanghai (CN); Jian LI, Shanghai (CN); Wei XIONG, Shanghai (CN)

(21) Appl. No.: 17/930,672

(22) Filed: Sep. 8, 2022

(30) Foreign Application Priority Data
Sep. 26, 2021 (CN) 202111128236.X

(51) Int. Cl. C12Q 1/6844; C12Q 1/70; C12Q 1/6888

(52) U.S. Cl. CPC C12Q 1/6844; C12Q 1/701; C12Q 1/6888; C12Q 2600/112

(57) ABSTRACT

The present disclosure provides a kit for detecting foot-and-mouth disease virus and a detection method thereof, and belongs to the technical field of biological detection. The kit includes crRNA, T7 transcriptase, NTP, a probe, Cas13a, and a nucleic acid amplification reagent; the nucleic acid amplification reagent includes a primer pair; the primer pair is selected from nucleic acid sequences shown in SEQ ID No: 1 and SEQ ID No: 2, and/or those shown in SEQ ID No: 3 and SEQ ID No: 4; and the crRNA is selected from nucleic acid sequences shown in SEQ ID No: 5 and SEQ ID No: 6. The kit can be used for detecting the foot-and-mouth disease virus for non-diagnostic treatment purposes; the method has extremely high specificity and sensitivity, and provides a reference for preparation and production of a detection reagent for major animal diseases based on isothermal amplification.

### CROSS REFERENCE TO RELATED APPLICATION

This patent application claims the benefit and priority of Chinese Patent Application No. 202111128236.X, filed on Sep. 26, 2021, the disclosure of which is incorporated by reference herein in its entirety as part of the present application.

### TECHNICAL FIELD

The present disclosure relates to the technical field of biological detection, in particular to a kit for detecting foot-and-mouth disease virus (FMDV) and a detection method thereof.

### BACKGROUND ART

Foot-and-mouth disease is an acute, malignant, highly contagious infectious disease caused by foot-and-mouth disease virus (FMDV) in cloven-hoofed animals such as pigs, cattle, and sheep. Once it breaks out, the infected and contagious animals must be culled. Foot-and-mouth disease is listed as a notifiable disease by the World Organization for Animal Health, and heeds the list of Category I animal diseases in China. It may not only cause huge direct economic losses, but also seriously endanger the sound development of animal husbandry and the foreign trade of related products, having far-reaching impact on politics and economy of a country.

Foot-and-mouth disease has 7 serotypes and more than 65 serosubtypes. There is no cross-immunity between serotypes, so it is difficult to control. Foot-and-mouth disease mainly includes 7 serotypes: serotypes A, O, C, Asial, and South Africa 1/2/3 (SAT1/2/3). Due to the high density of breeding animals, diseased animals exhale and excrete a large number of pathogenic microorganisms, resulting in the accumulation of high-concentration microbial aerosols in the house; moreover, through the exchange of gas inside and outside the house, pathogenic microorganisms spread and diffuse with the atmosphere, causing environmental microbial pollution and epidemic spreading, so there is a high demand for virus detection.

For virus detection, nucleic acid detection methods are usually used, but the current nucleic acid detection methods still have many deficiencies. Reverse transcription-polymerase chain reaction (RT-PCR) and fluorescent RT-PCR require amplification equipment with specific temperature cycles, which are not convenient for on-site quarantine. Currently widely used isothermal amplification technologies such as loop-mediated isothermal amplification (LAMP) and recombinase aided amplification (RAA) can meet the needs for on-site detection. However, LAMP may easily lead to false positivity due to its defect in specificity. RAA is very sensitive to the design of primers and needs to be screened from multiple primer pairs to meet the requirements for high-sensitivity detection.

Therefore, it is of great significance to develop a new method for detecting FMDV while meeting the needs of convenient on-site quarantine and detection sensitivity.

### SUMMARY

The present disclosure aims to solve at least one of the technical problems existing in the prior art. To this end, the present disclosure provides a kit for detecting FMDV, which can perform detection under constant temperature conditions, reduces dependence on fluorescent PCR detection equipment, and has high detection sensitivity.

The present disclosure further provides a method of use of the foregoing kit.

The present disclosure further provides use of the foregoing kit in the detection of FMDV for non-diagnostic treatment purposes.

According to one aspect of the present disclosure, a kit for detecting FMDV is provided, and the kit includes: crRNA, T7 transcriptase, NTP, a probe, Cas13a, and a nucleic acid amplification reagent;

the nucleic acid amplification reagent includes a primer pair, and the primer pair is selected from nucleic acid sequences shown in SEQ ID No: 1 and SEQ ID No: 2, and/or those shown in SEQ ID No: 3 and SEQ ID No: 4; the crRNA is selected from nucleic acid sequences shown in SEQ ID No: 5 and SEQ ID No: 6.

In the present disclosure, the primer pair shown in SEQ ID No: 1 and SEQ ID No: 2 and the crRNA shown in SEQ ID No: 5 are used to detect FMDV serotypes Asianl, SAT1/2/3 and C strains; the primer pair shown in SEQ ID No: 3 and SEQ ID No: 4 and the crRNA shown in SEQ ID No: 6 are used to detect FMDV serotypes O and A strains. Due to a large variation of a virus sequence, two sets of primers are used to achieve full coverage of the detection of different subtypes of FMDV strains.

According to a specific example of the present disclosure, there are at least the following beneficial effects: the kit may realize high-sensitivity detection by a method for binding Cas13a by isothermal amplification; herein, the Cas13a can be activated by protein recognition and binding a target RNA sequence thereof, activated Cas13a can decompose non-target RNAs in the environment therein, and Cas13a-mediated non-specific cleavage of reporter RNA can be used to detect an isothermally amplified target nucleic acid sequence. The advantage of this detection technology is mainly that detection results feature low variability and high sensitivity.

In some examples of the present disclosure, the probe is FAM-UUUUUUUUUUUUUU-TAMRA (SEQ ID NO: 9), where the FAM and the TAMRA are fluorophores. In other examples, the fluorophore may further be at least selected from the group consisting of TET, HEX, Cy3, Cy5, and ROX. The probe may be applied to all detection systems of the present disclosure, and have a wide range of application and high sensitivity.

In some examples of the present disclosure, the nucleic acid amplification reagent may further include an amplification reagent for recombinase polymerase amplification (RPA) or recombinase-aid amplification (RAA).

In the present disclosure, both the recombinase polymerase amplification (RPA) and the recombinase-aid amplification (RAA) are commonly used isothermal amplification technologies, and during amplification, both technologies use recombinase, single-stranded binding protein, and DNA polymerase to perform rapid nucleic acid amplification at a constant temperature of 37° C. A difference lies in the source of the recombinase. The recombinase of the RPA system is derived from T4 phage, and the recombinase of the RAA system is derived from bacteria or fungi. In some examples of the present disclosure, commercially available nucleic acid amplification reagents for the RPA or RAA system may be purchased for reactions.

In some examples of the present disclosure, the crRNA may have a concentration of 10 μM, the T7 transcriptase may have concentration of 1 mg/mL, the NTP may have a concentration of 100 mM, the probe may have a concentration of 10 μM , the Cas13a may have a concentration of 1 mg/mL. Each of the above concentration may be a final concentration of each component in the system.

In some examples of the present disclosure, the kit may further include: a reaction buffer and water.

In some preferred examples of the present disclosure, the reaction buffer may include a pH 7.3 buffer containing 40 mM Tris-HCl, 60 mM NaCl, and 6 mM MgCl₂ at final concentrations thereof.

In some preferred examples of the present disclosure, the kit may include: 1 μL of the crRNA, 1.5 μL of the T7 transcriptase, 4 μL of the NTP, 2.5 μL of the probe, 0.5 μL of the Cas13a, 2.5 μL of a nucleic acid amplification product, 2.5 μL of the reaction buffer, and 11 μL of the water; the nucleic acid amplification product may be amplified by the nucleic acid amplification reagent.

In some more preferred examples of the present disclosure, the kit may include: 1 μL of the crRNA (concentration 10 μM), 1.5 μL of the T7 transcriptase (concentration 1 mg/mL), 4 μL of the NTP (concentration 100 mM), 2.5 μL of the probe (concentration 10 μM), 0.5 μL of the Cas13a (concentration 1 mg/mL), 2.5 μL of the nucleic acid amplification product, 2.5 μL of the reaction buffer, and 11 μL of the water; the nucleic acid amplification product may be amplified by the RPA or RAA system, and/or purified by a phenol-chloroform method. Each of the above concentration may be a final concentration of each component in the system.

According to another aspect of the present disclosure, a method of use of the kit is provided, including the following steps:

step S1, obtaining a nucleic acid amplification product by using the nucleic acid amplification reagent;

step S2, mixing the nucleic acid amplification product with the crRNA, the T7 transcriptase, the NTP, the probe and the Cas13a in proportion to obtain a reaction solution; and

step S3, reacting the reaction solution and collecting fluorescence.

The method of use according to a specific example of the present disclosure has at least the following beneficial effects: the method is used to detect FMDV, can be performed under isothermal conditions, reduces dependence on fluorescent PCR detection equipment, and has higher detection sensitivity; this method enriches clinical detection methods and provides a reference for the preparation and production of detection reagents for major animal diseases based on isothermal amplification.

In some examples of the present disclosure, in step S1, the nucleic acid amplification product obtained by the nucleic acid amplification reagent may include: a nucleic acid amplification product amplified by the RPA or RAA system; and/or a nucleic acid amplification product purified by the phenol-chloroform method.

In some examples of the present disclosure, the reaction solution in step S2 may include: 1 μL of the crRNA, 1.5 μL of the T7 transcriptase, 4 μL of the NTP, 2.5 μL of the probe, 0.5 μL of the Cas13a, 2.5 μL of the nucleic acid amplification product, 2.5 μL of a reaction buffer, and 11 μL of water.

In some examples of the present disclosure, in step S3, reacting the reaction solution and collecting the fluorescence may include the following conditions: a temperature at which the reaction is carried out is 39° C.; and/or time for collecting the fluorescence is 30-120 min.

According to another aspect of the present disclosure, use of the kit in the detection of FMDV for non-diagnostic treatment purposes is provided.

The present disclosure has at least the following beneficial effects: the kit provided by the present disclosure may realize high-sensitivity detection by a method for binding Cas13a by isothermal amplification; the kit and the detection method may be used to detect FMDV, be performed under isothermal conditions, reduce dependence on fluorescent PCR detection equipment, and have higher detection sensitivity; this method may enrich clinical detection methods and provide a reference for the preparation and production of detection reagents for major animal diseases based on isothermal amplification.

In the description of this specification, the description with reference to the terms “one example/implementation”, “some examples/implementations”, and the like means that the specific features, structures, materials or characteristics described with reference to the implementation(s) or example(s) are included in at least one example of the present disclosure. In this specification, the schematic representations of the above-mentioned terms do not necessarily refer to the same embodiment or example. Moreover, the described specific features, structures, materials or characteristics can be combined in any one or more embodiments or examples in a suitable manner.

### BRIEF DESCRIPTION OF THE DRAWINGS

The present disclosure will be further described below with reference to accompanying drawings and examples, herein:

FIG. 1 illustrates agarose gel electrophoresis of the RAA method at different concentrations of plasmids of FMDV serotypes Asian1, SAT1/2/3, and C strains in an example of the present disclosure;

FIG. 2 illustrates fluorescence detection of the RAA-Cas13a method at different concentrations of plasmids of FMDV serotypes Asian1, SAT1/2/3, and C strains in an example of the present disclosure;

FIG. 3 illustrates agarose gel electrophoresis of the RAA method at different concentrations of plasmids of FMDV serotypes O and A strains in an example of the present disclosure;

FIG. 4 illustrates fluorescence detection of the RAA-Cas13a method at different concentrations of plasmids of FMDV serotypes O and A strains in an example of the present disclosure;

FIG. 5 illustrates fluorescence detection of the RAA-Cas13a method for specifically detecting positive nucleic acids of FMDV serotypes Asian1, SAT1/2/3, and C strains and those of other 7 swine disease viruses in an example of the present disclosure;

FIG. 6 illustrates fluorescence detection of the RAA-Cas13a method for specifically detecting positive nucleic acids of FMDV serotypes O and A strains and those of other 7 swine disease viruses in an example of the present disclosure.

### DETAILED DESCRIPTION OF THE EMBODIMENTS

The concept of the present disclosure and the technical effects produced thereby will be described clearly and completely below with reference to the examples, so as to fully understand the purpose, features and effects of the present disclosure. Obviously, the described examples are only a part of, not all of, the examples of the present disclosure. Based on the examples of the present disclosure, all other examples obtained by those skilled in the art without creative efforts shall fall within the protection scope of the present disclosure. All test methods used in the examples are conventional methods, unless otherwise specified; all materials and reagents used are commercially available, unless otherwise specified.

Sources of reagents and materials: Cas13a enzyme is prepared, expressed and purified according to method in the literature (J. S. Gootenberg et al., Science, Nucleic acid detection with CRISPR-Cas13a/C2c2, 2017); RT-RAA detection kit is purchased from Hangzhou ZC Bio-Sci & Tech Co. Ltd.; RNaseAlert Lab Test Kit V2 is purchased from Invitrogen, and T7 kit is purchased from NEB. Other reagents are analytical reagents made in China.

### EXAMPLE

A kit for detecting FMDV was provided, including: crRNA, T7 transcriptase, NTP, a probe, Cas13a, a nucleic acid amplification reagent, a reaction buffer, and water.

Herein, the nucleic acid amplification reagent included a primer pair designed based on the FMDV. The sequences of the designed primer pair were: (1) SEQ ID No: 1 and SEQ ID No: 2, and crRNA sequence: SEQ ID No: 5, which were used to detect FMDV serotypes Asian1, SAT1/2/3, and C strains; (2) SEQ ID No: 3 and SEQ ID No: 4, and crRNA sequence: SEQ ID No: 6, which were used to detect FMDV serotypes O and A strains. The nucleic acid amplification reagents used also included other reagents for the RAA system. Due to a large variation of the virus sequence, two sets of primers were used in this example to achieve full coverage of the detection of different subtypes of FMDV strains.

The specific sequence information is shown in Table 1 below:

TABLE 1
Primers and crRNA sequences of FMDV

| Name | Sequence (5′-3′) |
|---|---|
| FMDVascF | AATTCTAATACGACTCACTATAGGGACTGGGTTTTACAAA |
| | CCTGTGATGGCTTCGAAGA (SEQ ID No. 1) |
| FMDVascR | CGCAGGTAAAGTGATCTGTAGCTTGGAATCTC (SEQ ID |
| | No. 2) |
| FMDVasc- | GAUUUAGACUACCCCAAAAACGAAGGGGACUAAAACUCCC |
| crRNA | ACGGCGUGCAAAGGAGAGGAUAGC |
| | (SEQ ID No. 5) |
| FMDVoaF | AATTCTAATACGACTCACTATAGGGGACTATGGAACTGGG |
| | TTTTACAAACCTGTGATGGC (SEQ ID No. 3) |
| FMDVoaR | GGCGTTCACCCAACGCAGGTAAAGTGATCTGTA(SEQ ID |
| | No. 4) |
| FMDVoa- | GAUUUAGACUACCCCAAAAACGAAGGGGACUAAAACUCCC |
| crRNA | ACGGCGUGCAAAGGAGAGGAUAGC |
| | (SEQ ID No. 6) |

crRNA was synthesized by using crDNA, herein, FMDVasc-crDNA sequence (SEQ ID No: 7) was: AATTCTAATACGACTCACTATAGGGGATTTAGACTACCCCAAAAACGAAGGGGACTAA AACTCCCACGGCGTGCAAAGGAGAGGATAGC; FMDVoa-crDNA sequence was: AATTCTAATACGACTCACTATAGGGGATTTAGACTACCCCAAAAACGAAGGGGACTAA AACTCCCACGGCGTGCAAAGGAGAGGATAGC (SEQ ID No: 8).

The probe used in this example was FAM-UUUUUUUUUUUUUU-TAMRA (SEQ ID NO: 9), where the FAM and the TAMRA are fluorophores. In other examples, the fluorophore may further be selected from group consisting of TET, HEX, Cy3, Cy5, and ROX. A method of use of a kit for detecting FMDV, namely an FMDV RAA-Cas13a detection method, was provided, including the following steps:

Step S1. A nucleic acid amplification product was obtained by the nucleic acid amplification reagent, and the system was composed of the designed primer pair and the amplification reagent for RAA, and the FMDV detection sample was used as a template for amplification.

Step S2. A reaction solution was prepared, and the restricted digestion reaction system (a 25 μL system) after isothermal amplification was constructed as follows: 2.5 μL of 10× reaction buffer, 1 μL of crRNA (10 μM), 1.5 μL of T7 transcriptase (1 mg/mL), 4 μL of NTP (100 mM), 2.5 μL of probe (10 μM), 2.5 μL of RAA product purified by the phenol-chloroform method, 11 μL of water, and 0.5 μL of Cas13a (1 mg/mL). Each of the above concentration was a final concentration of each component in the system. Herein, the reaction buffer contained reagents with the following components at final concentrations: 40 mM Tris-HCl, 60 mM NaCl, and 6 mM MgCl₂, constituting a pH 7.3 buffer system.

S3. The reaction solution system prepared in step S2 was reacted and fluorescence was collected. The reaction temperature was 39° C., and the fluorescence was collected for 30 min.

In the present disclosure, the detection method in the example was referred to as “RAA-Cas13a detection” or “RAA-Cas13a method”.

### COMPARATIVE EXAMPLE

A RAA system kit (purchased from Hangzhou ZC Bio-Sci & Tech Co. Ltd.) was used in this comparative example, and the kit of the comparative example was used to detect FMDV. The specific process was as follows:

S1. An RAA amplification system was used; according to the 25 μL reaction system, the volume of each reagent component was in accordance with the kit instructions, the template volume was 1 μL, and the reaction conditions were implemented at 37° C. for 30 min.

Step S2, after RAA, the detection results were analyzed by agarose gel electrophoresis.

In the present disclosure, the detection method in the comparative example was referred to as “RAA detection” or “RAA method”.

### TEST EXAMPLE

The detection effects of the kits of the example and the comparative example on FMDV was tested in this test example (in the following test, the detection method in the example was referred to as “RAA-Cas13a detection” or “RAA-Cas13a method”, and that in the comparative example was referred to as “RAA detection” or “RAA method”), mainly including their detection sensitivity, specificity and accuracy. The specific experiments were as follows:

### 1. Sensitivity of RAA-Cas13a Detection

The detection was conducted according to the detection methods in the example and the comparative example, respectively:

(1) For different concentrations of plasmids of FMDV serotypes Asian1, SAT1/2/3, and C strains, at this time, the primer pair used was FMDVascF (SEQ ID No: 1) and FMDVascR (SEQ ID No: 2). Gradiently diluted plasmids were used as templates. The plasmid concentrations were 1×10⁶ copies/μL, 1×10⁵ copies/μL, 1×10⁴ copies/μL, 1×10³ copies/μL, 1×10² copies/μL, 1×10¹ copies/μL, 1×10⁰ copy/μL, and 0 copies/μL, respectively. Experimental results were obtained:

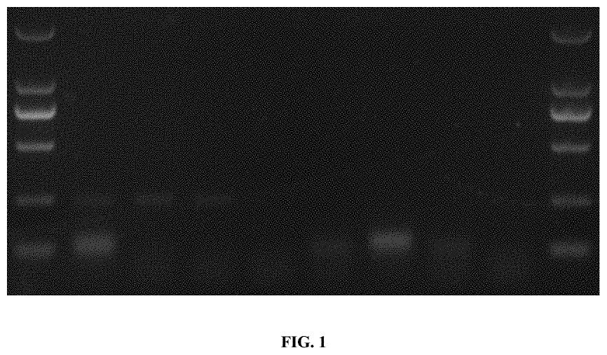

Comparative Example: For different concentrations of plasmids of FMDV serotypes Asian1, SAT1/2/3, and C strains, the minimum copy number of positive plasmids detected by ordinary RAA was 10⁴ copies/μL, but 10³ copies/μL plasmids and those and lower this concentration could not be detected. The results are shown in FIG. 1, where M represents DL2000 DNA Marker, and wells 1 to 8 represent plasmid concentrations of 1×10⁶ copies/μL, 1×10⁵ copies/μL, 1×10⁴ copies/μL, 1×10³ copies/μL, 1×10² copies/μL, 1×10¹ copies/μL, 1×10⁰ copy/μL, and 0 copies/μL, respectively.

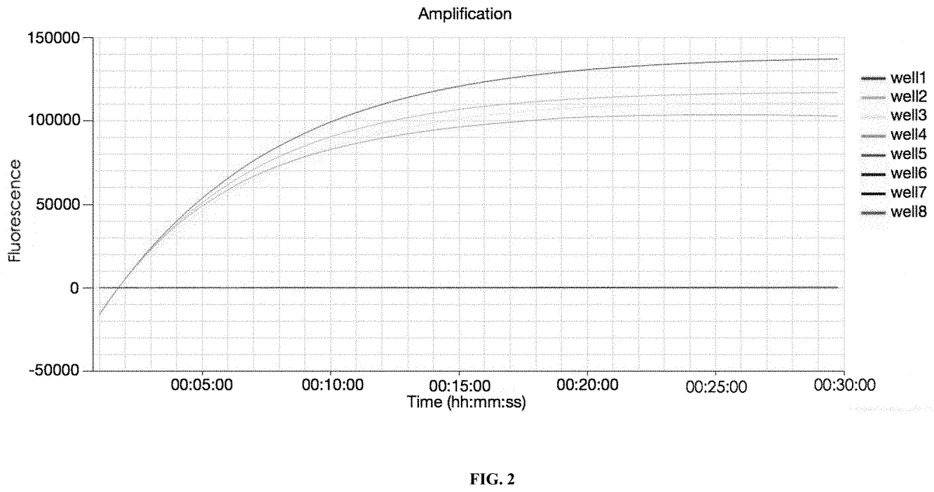

Implementaion Example: For different concentrations of plasmids of FMDV serotypes Asian1, SAT1/2/3, and C strains, the kit in the example was used for detection (RAA-Cas13a detection), and a minimum of 10² copies/μL positive plasmids could be detected. The results are shown in FIG. 2, where wells 1 to 8 represent plasmid concentrations of 1×10⁶ copies/μL, 1×10⁵ copies/μL, 1×10⁴ copies/μL, 1×10³ copies/μL, 1×10² copies/μL, 1×10¹ copies/μL, 1×10⁰ copy/μL, and 0 copies/μL, respectively.

(2) For different concentrations of plasmids of FMDV serotypes O and A strains, at this time, the primer pair used was FMDVoaF (SEQ ID No: 3) and FMDVascR (SEQ ID No: 4). Gradiently diluted plasmids were used as templates. The plasmid concentrations were 1×10⁶ copies/μL, 1×10⁵ copies/μL, 1×10⁴ copies/μL, 1×10³ copies/μL, 1×10² copies/μL, 1×10¹ copies/μL, 1×10⁰ copy/μL, and 0 copies/μL, respectively.

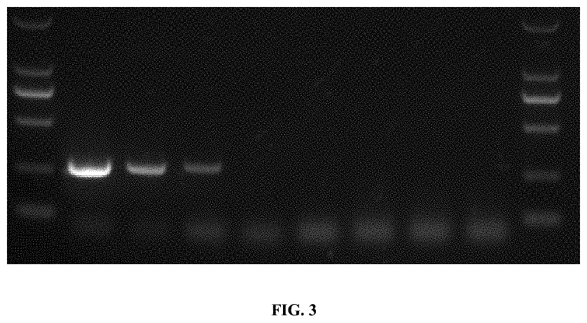

Comparative Example: For different concentrations of plasmids of FMDV serotypes O and A strains, the minimum copy number of positive plasmids detected by ordinary RAA was 10⁴ copies/μL, but 10³ copies/μL plasmids and those and lower this concentration could not be detected. The results are shown in FIG. 3, where M represents DL2000 DNA Marker, and wells 1 to 8 represent plasmid concentrations of 1×10⁶ copies/μL, 1×10⁵ copies/μL, 1×10⁴ copies/μL, 1×10³ copies/μL, 1×10² copies/μL, 1×10¹ copies/μL, 1×10⁰ copy/μL, and 0 copies/μL, respectively.

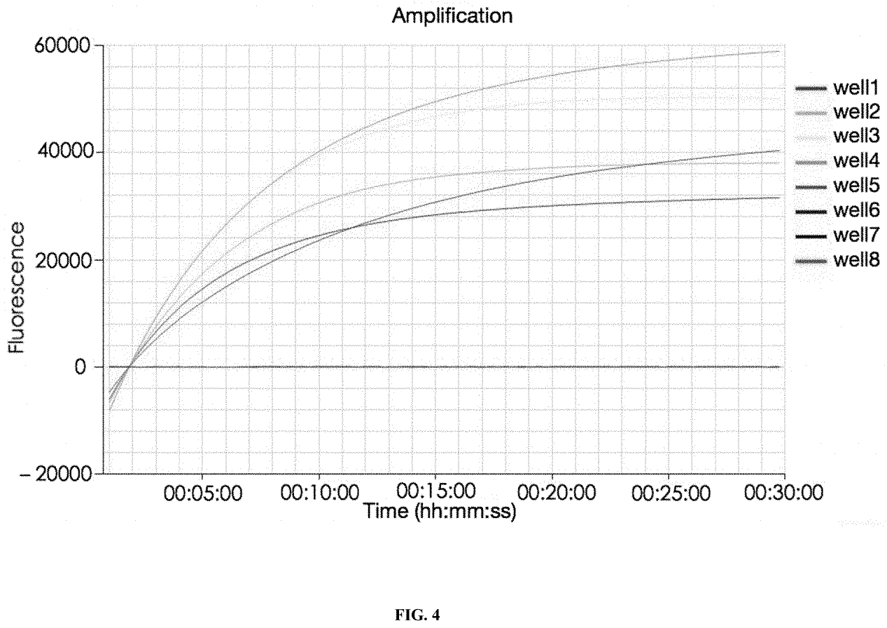

Implementaion Example: For different concentrations of plasmids of FMDV serotypes O and A strains, the kit in the example was used for detection (RAA-Cas13a detection system), and a minimum of 10² copies/μL positive plasmids could be detected. The results are shown in FIG. 4, where wells 1 to 8 represent plasmid concentrations of 1×10⁶ copies/μL, 1×10⁵ copies/μL, 1×10⁴ copies/μL, 1×10³ copies/μL, 1×10² copies/μL, 1×10¹ copies/μL, 1×10⁰ copy/μL, and 0 copies/μL, respectively.

### 2. Specificity of RAA-Cas13a Detection

In order to test the specificity of this method, positive nucleic acids of FMDV strains and those of other 7 swine disease viruses were detected by the RAA-Cas13a method. Specific steps were as follows:

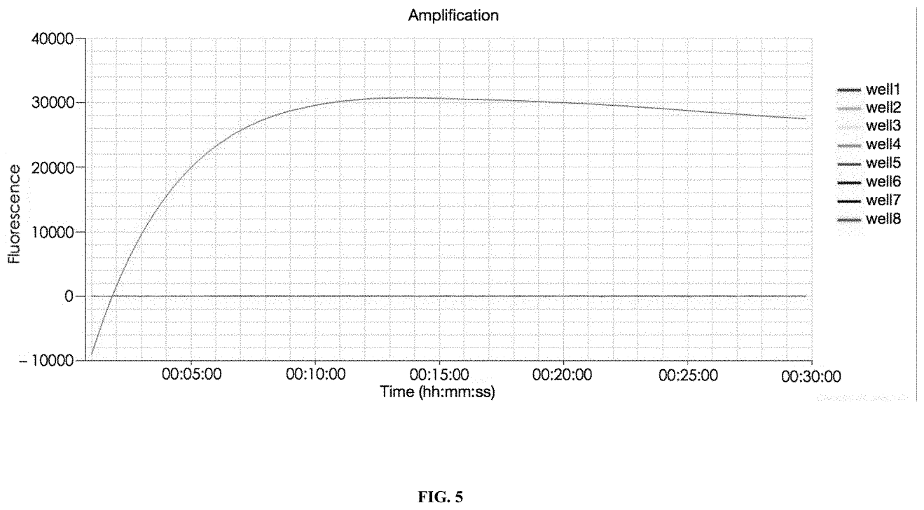

(1) A primer pair for serotypes Asian1, SAT1/2/3, and C (the primer pair used is shown in SEQ ID No: 1 and SEQ ID No: 2) was used to detect positive clinical samples. The results are shown in FIG. 5, where well 1 represents FMDV; well 2 represents porcine epidemic diarrhea virus (PEDV); well 3 represents transmissible gastroenteritis virus (TGEV); well 4 represents porcine reproductive and respiratory syndrome virus (PRRSV); well 5 represents porcine parvovirus (PPV); well 6 represents classical swine fever virus; well 7 represents African swine fever virus (ASFV); and well 8 represents porcine circovirus 2 (PCV2).

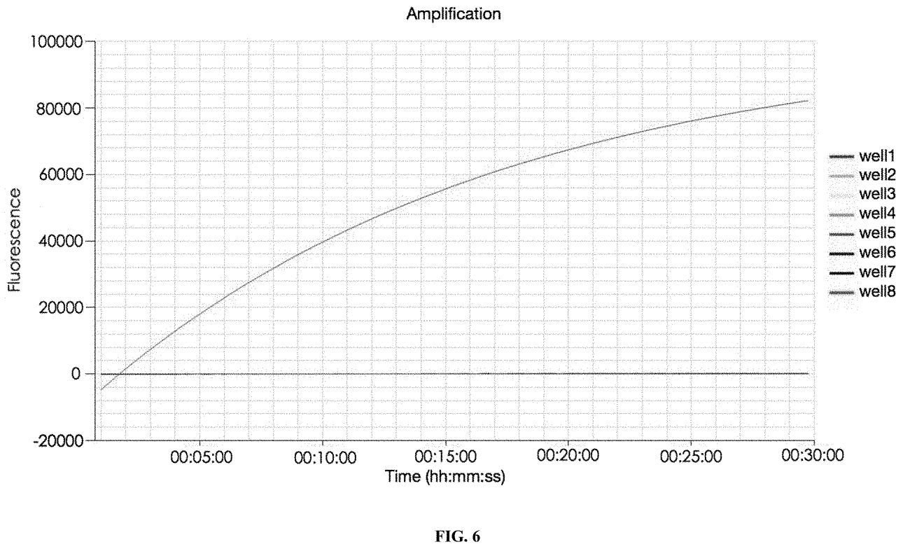

(2) A primer pair for serotypes O and A (the primer pair used is shown in SEQ ID No: 3 and SEQ ID No: 4) was used to detect positive clinical samples. The results are shown in FIG. 6, where well 1 represents FMDV; well 2 represents PEDV; well 3 represents TGEV; well 4 represents PRRSV; well 5 represents PPV; well 6 represents CSFV; well 7 represents ASFV; and well 8 represents PCV2.

After positive samples were amplified by RAA, obvious fluorescence signal enhancement of FMDV-positive nucleic acids could be detected by the Cas13a reaction system 10 min after the reaction, and the positive nucleic acids of the remaining 7 swine disease viruses had no fluorescence signal enhancement.

3. Consistency Comparison with Detection Results of Fluorescent RT-PCR

Two sets of primer pairs designed in Example 1 were used for simultaneous detection, and if one set was tested positive, it was determined to be positive for FMDV. In order to evaluate the consistency between the method established in this study and fluorescent RT-PCR, 14 FMDV-positive cDNA samples preserved in this laboratory and other 38 negative pork nucleic acid samples prepared by reverse transcription were tested. All samples were simultaneously tested in accordance with the GB/T 22915-2008 Protocol of Universal Fluorogenic RT-PCR for Foot and Mouth Disease Virus. Among them, 13 samples that were tested positive by the fluorescent RT-PCR method could be detected by RAA-Cas13a, and the κ value was between 0.81 and 1.00 (κ=0.95), indicating that the detection results of the RAA-Cas13a method established in this study were almost identical to the those stipulated by the national standard. The detection results are shown in Table 2 below:

TABLE 2
Consistency comparison with FMDV detection results
of RAA-Cas13a method versus fluorescent RT-PCR

| | | Fluorescent RT-PCR | | |
|---|---|---|---|---|
| | | Positive | Negative | Total |
| RAA-Cas13a | Positive | 13 | 0 | 13 |
| | Negative | 1 | 29 | 30 |
| | Total | 14 | 29 | 43 |

Compared with the fluorescent RT-PCR method in the standard, the specificity of the RAA-Cas13a method established in this study was: 29/(29+0)=100.00%; the sensitivity was: 13/(13+1)=92.86%; Po=(13+29)/43=97.67%; Pe=14/43×13/43+29/43×30/43=56.90%; κ=(Po−Pe)/(1−Pe)=0.95.

In summary, the kit of the present disclosure has extremely high specificity and sensitivity for the detection of FMDV. Two sets of primers can be used to detect a total of seven FMDV serotypes, namely, serotypes A, O, C, Asial, and SA1/2/3, and have low demand for the template concentration, while a minimum of 10² copies/μL positive plasmids can be detected. Therefore, the kit and the detection method provided by the present disclosure are used for the detection of foot-and-mouth disease virus, enrich clinical detection methods, and provide a reference for the preparation and production of detection reagents for major animal diseases based on isothermal amplification.

The examples of the present disclosure have been described in detail above with reference to the accompanying drawings, but the present disclosure is not limited thereto. Within the scope of knowledge possessed by those of ordinary skill in the art, various modifications can be made without departing from the purpose of the present disclosure. In addition, in the case of no conflict, the examples of the present disclosure and the features in the examples may be combined with each other.

### Sequence Listing Information:

DTD Version: V1_3
File Name: GWP20220700074-Sequencing Lising.xml
Software Name: WIPO Sequence
Software Version: 2.1.2
Production Date: 2022-09-02

### General Information:

Current application/Applicant file reference: GWP20220700074
Earliest priority application/IP Office: CN
Earliest priority application/Application number: 202111128236.X
Earliest priority application/Filing date: 2021-09-26
Applicant name: AFTC of Shanghai Customs
Applicant name/Language: en
Invention title: KIT FOR DETECTING FOOT-AND-MOUTH DISEASE VIRUS AND DETECTION METHOD THEREOF (en)
Sequence Total Quantity: 9

Sequences:

| | |
|---|---|
| Sequence Number (ID): 1 | |
| Length: 59 | |
| Molecule Type: DNA | |
| Features Location/Qualifiers: | |
| - source, 1 . . . 59 | |
| >PCR_primers, | |
| fwd_name: FMDVascF, fwd_seq: aattctaatacgactcactatagggactgggttttacaaacctgtgatggcttcgaaga, | |
| rev_name: FMDVascR, rev_seq: cgcaggtaaagtgatctgtagcttggaatctc | |
| > mol_type, other DNA | |
| > note, Primer_FMDVascF | |
| > organism, synthetic construct | |
| Residues: | |
| aattctaata cgactcacta tagggactgg gttttacaaa cctgtgatgg cttcgaaga | 59 |
| Sequence Number (ID): 2 | |
| Length: 32 | |
| Molecule Type: DNA | |
| Features Location/Qualifiers: | |
| - source, 1 . . . 32 | |
| >PCR_primers, | |
| fwd_name: FMDVascF, fwd_seq: aattctaatacgactcactatagggactgggttttacaaacctgtgatggcttcgaaga, | |
| rev_name: FMDVascR, rev_seq: cgcaggtaaagtgatctgtagcttggaatctc | |
| > mol_type, other DNA | |
| > note, Primer_FMDVascR | |
| > organism, synthetic construct | |
| Residues: | |
| cgcaggtaaa gtgatctgta gcttggaatc tc | 32 |
| Sequence Number (ID): 3 | |
| Length: 60 | |
| Molecule Type: DNA | |
| Features Location/Qualifiers: | |
| - source, 1 . . . 60 | |
| >PCR_primers, | |
| fwd_name: FMDVoaF, fwd_seq: aattctaatacgactcactataggggactatggaactgggttttacaaacctgtgatggc, | |
| rev_name: FMDVoaR, rev_seq: ggcgttcacccaacgcaggtaaagtgatctgta | |
| > mol_type, other DNA | |
| > note, Primer_FMDVoaF | |
| > organism, synthetic construct | |
| Residues: | |
| aattctaata cgactcacta taggggacta tggaactggg ttttacaaac ctgtgatggc | 60 |
| Sequence Number (ID): 4 | |
| Length: 33 | |
| Molecule Type: DNA | |
| Features Location/Qualifiers: | |
| - source, 1 . . . 33 | |
| >PCR_primers, | |
| fwd_name: FMDVoaF, fwd_seq: aattctaatacgactcactataggggactatggaactgggttttacaaacctgtgatggc, | |
| rev_name: FMDVoaR, rev_seq: ggcgttcacccaacgcaggtaaagtgatctgta | |
| > mol_type, other DNA | |
| > note, Primer_FMDVoaR | |
| > organism, synthetic construct | |
| Residues: | |
| ggcgttcacc caacgcaggt aaagtgatct gta | 33 |
| Sequence Number (ID): 5 | |
| Length: 64 | |
| Molecule Type: RNA | |
| Features Location/Qualifiers: | |
| - source, 1 . . . 64 | |
| > mol_type, other RNA | |
| > note, crRNA sequence: FMDVasc-crRNA | |
| > organism, synthetic construct | |
| Residues: | |
| gatttagact accccaaaaa cgaaggggac taaaactccc acggcgtgca aaggagagga | 60 |
| tagc | 64 |
| Sequence Number (ID): 6 | |
| Length: 64 | |
| Molecule Type: RNA | |
| Features Location/Qualifiers: | |
| - source, 1 . . . 64 | |
| > mol_type, other RNA | |
| >note, crRNA sequence: FMDVoa-crRNA | |
| > organism, synthetic construct | |
| Residues: | |
| gatttagact accccaaaaa cgaaggggac taaaactccc acggcgtgca aaggagagga | 60 |
| tagc | 64 |
| Sequence Number (ID): 7 | |
| Length: 89 | |
| Molecule Type: DNA | |
| Features Location/Qualifiers: | |
| - source, 1 . . . 89 | |
| > mol_type, other DNA | |
| > note, FMDVasc-crDNA sequence | |
| > organism, synthetic construct | |
| Residues: | |
| aattctaata cgactcacta taggggattt agactacccc aaaaacgaag gggactaaaa | 60 |
| ctcccacggc gtgcaaagga gaggatagc | 89 |
| Sequence Number (ID): 8 | |
| Length: 89 | |
| Molecule Type: DNA | |
| Features Location/Qualifiers: | |
| - source, 1 . . . 89 | |
| > mol_type, other DNA | |
| > note, FMDVoa-crDNA sequence | |
| > organism, synthetic construct | |
| Residues: | |
| aattctaata cgactcacta taggggattt agactacccc aaaaacgaag gggactaaaa | 60 |
| ctcccacggc gtgcaaagga gaggatagc | 89 |
| Sequence Number (ID): 9 | |
| Length: 14 | |
| Molecule Type: RNA | |
| Features Location/Qualifiers: | |
| - source, 1 . . . 14 | |
| > mol_type, other RNA | |
| > note, sequence of probe | |
| > organism, synthetic construct | |
| Residues: | |
| tttttttttt tttt | 14 |
| END | |

What is claimed:

1. A kit for detecting foot-and-mouth disease virus, wherein the kit comprises: crRNA, T7 transcriptase, NTP, a probe, Cas13a, and a nucleic acid amplification reagent;
the nucleic acid amplification reagent comprises a primer pair, and the primer pair is selected from nucleic acid sequences shown in SEQ ID No: 1 and SEQ ID No: 2, and/or those shown in SEQ ID No: 3 and SEQ ID No: 4;
the crRNA is selected from nucleic acid sequences shown in SEQ ID No: 5 and SEQ ID No: 6.

2. The kit according to claim 1, wherein the nucleic acid amplification reagent further comprises an amplification reagent for recombinase polymerase amplification (RPA) or recombinase-aid amplification (RAA).

3. The kit according to claim 1, wherein the crRNA has a concentration of 10 μM, the T7 transcriptase has concentration of 1 mg/mL, the NTP has a concentration of 100 mM, the probe has a concentration of 10 μM , the Cas13a has a concentration of 1 mg/mL.

4. The kit according to claim 1, wherein the kit further comprises: a reaction buffer and water.

5. The kit according to claim 4, wherein the kit comprises: 1 μL of the crRNA, 1.5 μL of the T7 transcriptase, 4 μL of the NTP, 2.5 μL of the probe, 0.5 μL of the Cas13a, 2.5 μL of a nucleic acid amplification product, 2.5 μL of the reaction buffer, and 11 μL of the water; the nucleic acid amplification product is amplified by the nucleic acid amplification reagent.

6. A method of use of the kit according to claim 1, comprising the following steps:
step S1, obtaining a nucleic acid amplification product by using the nucleic acid amplification reagent;
step S2, mixing the nucleic acid amplification product with the crRNA, the T7 transcriptase, the NTP, the probe and the Cas13a in proportion to obtain a reaction solution; and
step S3, reacting the reaction solution and collecting fluorescence.

7. The method of use according to claim 6, wherein the nucleic acid amplification reagent further comprises an amplification reagent for recombinase polymerase amplification (RPA) or recombinase-aid amplification (RAA).

8. The method of use according to claim 6, wherein the crRNA has a concentration of 10 μM, the T7 transcriptase has concentration of 1 mg/mL, the NTP has a concentration of 100 mM, the probe has a concentration of 10 μM, the Cas13a has a concentration of 1 mg/mL.

9. The method of use according to claim 6, wherein the kit further comprises: a reaction buffer and water.

10. The method of use according to claim 9, wherein the kit comprises: 1 μL of the crRNA, 1.5 μL of the T7 transcriptase, 4 μL of the NTP, 2.5 μL of the probe, 0.5 μL of the Cas13a, 2.5 μL of a nucleic acid amplification product, 2.5 μL of the reaction buffer, and 11 μL of the water; the nucleic acid amplification product is amplified by the nucleic acid amplification reagent.

11. The method of use according to claim 6, wherein, in step S1, the nucleic acid amplification product obtained by the nucleic acid amplification reagent comprises:
a nucleic acid amplification product amplified by an RPA or RAA system; and/or a nucleic acid amplification product purified by a phenol-chloroform method.

12. The method of use according to claim 7, wherein, in step S1, the nucleic acid amplification product obtained by the nucleic acid amplification reagent comprises:
a nucleic acid amplification product amplified by an RPA or RAA system; and/or a nucleic acid amplification product purified by a phenol-chloroform method.

13. The method of use according to claim 8, wherein, in step S1, the nucleic acid amplification product obtained by the nucleic acid amplification reagent comprises:
a nucleic acid amplification product amplified by an RPA or RAA system; and/or a nucleic acid amplification product purified by a phenol-chloroform method.

14. The method of use according to claim 6, wherein the reaction solution in step S2 comprises: 1 μL of the crRNA, 1.5 μL of the T7 transcriptase, 4 μL of the NTP, 2.5 μL of the probe, 0.5 μL of the Cas13a, 2.5 μL of the nucleic acid amplification product, 2.5 μL of a reaction buffer, and 11 μL of water.

15. The method of use according to claim 6, wherein, in step S3, reacting the reaction solution and collecting the fluorescence comprises the following conditions:
a temperature at which the reaction is carried out is 39° C.; and/or
time for collecting the fluorescence is 30-120 min.

16. Use of the kit according to claim 1 in the detection of foot-and-mouth disease virus for non-diagnostic treatment purposes.

17. The use according to claim 16, wherein the nucleic acid amplification reagent further comprises an amplification reagent for recombinase polymerase amplification (RPA) or recombinase-aid amplification (RAA).

18. The use according to claim 16, wherein the crRNA has a concentration of 10 μM, the T7 transcriptase has concentration of 1 mg/mL, the NTP has a concentration of 100 mM, the probe has a concentration of 10 μM , the Cas13a has a concentration of 1 mg/mL.

19. The use according to claim 16, wherein the kit further comprises: a reaction buffer and water.

20. The use according to claim 19, wherein the kit comprises: 1 μL of the crRNA, 1.5 μL of the T7 transcriptase, 4 μL of the NTP, 2.5 μL of the probe, 0.5 μL of the Cas13a, 2.5 μL of a nucleic acid amplification product, 2.5 μL of the reaction buffer, and 11 μL of the water; the nucleic acid amplification product is amplified by the nucleic acid amplification reagent.


SEQUENCE LISTING (9 sequences)

SEQ ID NO: 1 - DNA, 59 bp, synthetic construct
  source 1..59: PCR_primers = fwd_name:FMDVascF,fwd_seq:aattctaatacgactcac tatagggactgggttttacaaacctgtgatggcttcgaaga,rev_name:FMDVasc R,rev_seq:cgcaggtaaagtgatctgtagcttggaatctc; mol_type = other DNA; note = Primer_FMDVascF
aattctaatacgactcactatagggactgggttttacaaacctgtgatggcttcgaaga

SEQ ID NO: 2 - DNA, 32 bp, synthetic construct
  source 1..32: PCR_primers = fwd_name:FMDVascF,fwd_seq:aattctaatacgactcac tatagggactgggttttacaaacctgtgatggcttcgaaga,rev_name:FMDVasc R,rev_seq:cgcaggtaaagtgatctgtagcttggaatctc; mol_type = other DNA; note = Primer_FMDVascR
cgcaggtaaagtgatctgtagcttggaatctc

SEQ ID NO: 3 - DNA, 60 bp, synthetic construct
  source 1..60: PCR_primers = fwd_name:FMDVoaF,fwd_seq:aattctaatacgactcact ataggggactatggaactgggttttacaaacctgtgatggc,rev_name:FMDVoaR ,rev_seq:ggcgttcacccaacgcaggtaaagtgatctgta; mol_type = other DNA; note = Primer_FMDVoaF
aattctaatacgactcactataggggactatggaactgggttttacaaacctgtgatggc

SEQ ID NO: 4 - DNA, 33 bp, synthetic construct
  source 1..33: PCR_primers = fwd_name:FMDVoaF,fwd_seq:aattctaatacgactcact ataggggactatggaactgggttttacaaacctgtgatggc,rev_name:FMDVoaR ,rev_seq:ggcgttcacccaacgcaggtaaagtgatctgta; mol_type = other DNA; note = Primer_FMDVoaR
ggcgttcacccaacgcaggtaaagtgatctgta

SEQ ID NO: 5 - RNA, 64 nt, synthetic construct
  source 1..64: mol_type = other RNA; note = crRNA sequence:FMDVasc-crRNA
gatttagactaccccaaaaacgaaggggactaaaactcccacggcgtgcaaaggagaggatagc

SEQ ID NO: 6 - RNA, 64 nt, synthetic construct; identical to SEQ ID NO: 5
  source 1..64: mol_type = other RNA; note = crRNA sequence:FMDVoa-crRNA

SEQ ID NO: 7 - DNA, 89 bp, synthetic construct
  source 1..89: mol_type = other DNA; note = FMDVasc-crDNA sequence
aattctaatacgactcactataggggatttagactaccccaaaaacgaaggggactaaaactcccacggcgtgcaaaggagaggatagc

SEQ ID NO: 8 - DNA, 89 bp, synthetic construct; identical to SEQ ID NO: 7
  source 1..89: mol_type = other DNA; note = FMDVoa-crDNA sequence

SEQ ID NO: 9 - RNA, 14 nt, synthetic construct
  source 1..14: mol_type = other RNA; note = sequence of probe
tttttttttttttt